(19) United States
(12) Patent Application Publication
(10) Pub. No.: US 2005/0123574 A1
(43) Pub. Date: Jun. 9, 2005

(54) Massaging toilet bar with disintegrable agglomerates

(75) Inventors: Syed Husain Abbas, Seymour, CT (US); Alessandro Luigi Spadini, Stamford, CT (US); Melissa Iva Katz, Weston, CT (US); Evan Hillman, Torrington, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 10/730,635

(22) Filed: Dec. 5, 2003

(51) Int. Cl. A61K 7/075; A61K 7/08

(52) U.S. Cl. 424/401; 424/70.22

(57) ABSTRACT

Massaging toilet bar compositions are described that contain disintegrable agglomerates that provide simultaneous exfoliation and massaging to the skin and hair. Agglomerates are made by treating them with hydrophilic liquids, hydrophobic liquids, or a combination thereof. This treatment makes the agglomerate softer but not so soft as to make it break apart during the manufacture of the bars.

### BACKGROUND OF THE INVENTION

2. Field of the Invention

The present invention relates to a massaging toilet bar with disintegrable agglomerates suitable for topical application for massaging and cleansing the human body, such as the skin and hair.

2. The Related Art

Exfoliating toilet bars are well known. However, the majority of them are very irritating to the skin due to the fact that they have high levels of harsh or marginally effective exfoliants. Examples of such prior art toilet bars with exfoliant particles or beads such as polyethylene beads, silica, walnut shells, and apricot seeds, and the like are described in e.g. U.S. Pat. Nos. 6,376,441; 6,342,470; 6,384,000; and 6,074,998. These exfoliating particles are usually very hard and impart an undesirable abrasive feel. Prior art agglomerates of such particles are also frequently sharp and scratchy. Surprisingly it has been found that disintegrable agglomerates can be formulated into toilet bars such as soap (including translucent glycerine type) bars, combo (syndet/soap) bars, syndet type bars or melt cast type bars. Such agglomerates can be made and used as massaging components in such bars by pre-treating them with hydrophilic or hydrophobic liquids that are preferably anhydrous. These inventive massaging bars will provide the user with the benefits of a gentle massage and increased blood circulation, and reduction in muscular tension which would thus improve the users overall well-being. Integral to the massage experience is the lubrication from the hydrophilic/hydrophobic liquid used to treat the inventive agglomerates. It is expected that additional deposition on the skin of the hydrophilic/hydrophobic liquid from the treated agglomerate will occur. Furthermore the treatment liquid helps in the moisturization and skin feel during use of the inventive bar. Surprisingly it was found that pre-treatment allows integration of relatively large particles that would otherwise be very harsh. Such large but gentle particles provide unique skin feel that cannot be obtained with smaller and typically harsher particles.

### SUMMARY OF THE INVENTION

In one aspect of the invention is a massaging toilet bar, including but not limited to:
a. about 5 to 80% by wt. of one or more anionic surfactants;
b. about 0.02 to 20% by wt. of disintegrable agglomerates, the agglomerates including a plurality of particles, optionally containing a binder; and a treatment fluid coating at least a portion of an interior surface of the particles in the agglomerates, the fluid being selected from a hydrophilic compound, a hydrophobic compound or a blend thereof;
c. wherein the agglomerates have a fracture index less than 1.0; and
d. wherein the bar has a rigidity index of greater than 0.2 Mpa at 25 C.

In another aspect of the invention is a process for manufacturing a massaging bar, including but not limited to the steps of in no particular order:
a. contacting particle agglomerates with a treatment fluid under conditions effective for the agglomerate to have attained a fracture index of less than 1.0 to form a pretreated agglomerate, the fluid being selected from a hydrophilic compound, a hydrophobic compound or a blend thereof;
b. blending the pretreated agglomerate with at least one component of a toilet bar base selected from a soap base, a syndet base, or a combar base, optionally containing a fragrance and other minor components to form a mixed mass;
c. treating the mixed mass via an effective combination of milling, refining and/or plodding to form a refined mass; and
d. processing the refined mass by either extruding followed by stamping, cutting or a combination thereof; or casting followed by hardening to form a massaging bar.

In a further aspect of the present inveniton is a method of skin cleansing with a massaging bar, including but not limited to the steps of:
a. wetting a massaging bar with water; the bar including:
   1. about 5 to 80% by wt. of one or more anionic surfactants;
   2. about 0.02 to 20% by wt. of water-disintegrable agglomerates, the agglomerates including a plurality of particles, optionally containing a binder; and a treatment fluid coating at least a portion of an interior surface of the particles in the agglomerates, the fluid being selected from a hydrophilic compound, a hydrophobic compound or a blend thereof;
   3. wherein the agglomerates have a fracture index less than 1.0; and
   4. wherein the bar has a rigidity index of greater than 0.2 Mpa at 25 C; and
b. rubbing the wet bar on the skin with sufficient force to cause the agglomerates to disintegrate while cleansing and massaging the skin.

### DETAILED DESCRIPTION OF THE INVENTION

In one aspect of the invention is a massaging toilet bar, including but not limited to:
a. about 5 to 80% by wt. of one or more anionic surfactants;
b. about 0.02 to 20% by wt. of disintegrable agglomerates, the agglomerates including a plurality of particles, optionally containing a binder; and a treatment fluid coating at least a portion of an interior surface of the particles in the agglomerates, the fluid being selected from a hydrophilic compound, a hydrophobic compound or a blend thereof (preferably the disintegrable agglomerates contain a minimum of about 0.05, 0.1, 0.2, 0.3 0.5 and a maxiumum of about 1.0, 1.1, 1.5, 2, 3, 5, 6, 7, 10, 11, 15, or 20% by wt. of the treatment fluid based on the aggomerate;
c. wherein the agglomerates have a fracture index less than 1.0 (preferably 0.9, 0.8, 0.7, 0.6, 0.5 or less); and
d. wherein the bar has a rigidity index of greater than 0.2 Mpa at 25 C.

The hardness of the granules before and after wetting in the benefit components is expressed as fracture index number. Fracture index number is defined as the ratio of the pressure applied (in grams) to break the pre-wet granule as compared to the pressure needed to break a non-treated granule. The fracture index for the different types of granules should be less than 1.0. Ideally it should be from 0.5-0.9. Fracture index number may be obtained with the fracture index method defined below. It was found that useful pre-treated agglomerates with a diameter of 0.3-1.0 mm had a fracture index of about 0.9 and pre-treated agglomerates with a diameter of 1.4-2.0 mm had a fracture index of about 0.7.

Both hydrophilic and hydrophobic beads may be used to obtain the desired fracture index. Hydrophilic beads preferably require a treatment fluid or medium of dispersion which is hydrophobic and the polarity of the hydrophobic medium will be observed to have large effect on the Fracture Index number. The desired Fracture Index number can be achieved by changing the polarity of the medium by selecting different liquids or by mixing 2 or more liquids of different polarity and the like. Examples of such beads are unmodified clay agglomerates, starch derivatives and the like. Hydrophobic Beads preferably require a dispersion medium that is hydrophilic such as water, alcohols, polyols, PEGs, alkanolamines such as TEA, and the like. The agglomerates of these beads may also be hydrophobically modified. Such modifications can be made to clay, cellulose materials or other starch derivatives and the like. Additionally blends of any of these two types of beads could be used to obtain a desired Fracture Index by mixing appropriate level of hydrophilic and hydrophobic liquids with different polarity.

A preferred embodiment of a method for wetting the agglomerates is as follows:
1. Combine agglomerates and treatment fluid
2. Mix to make sure all agglomerates are soaked in fluid
3. Let agglomerates soak for a minimum of 20 minutes
4. Once agglomerates have soaked add them to chip mixer with soap base
5. Add fragrance and other goodies to mixer
6. Send to plodder to be extruded

Advantageously the disintegrable agglomerates are disintegrable in water or any other hydrophilic substance with or without applied shear force. Preferably the average weight ratio of treatment fluid to particles and optional binder in the agglomerates is in the range of about 1:5 to about 5:1; more preferably 1:4 to 1:2. Preferably the average diameter of the agglomerates is in the range of about 0.1 to 20.0 mm (preferably with a minimum diameter of 0.2, 0.3, 0.5, 0.7, 1.0, and 2.0 mm and a maximum diameter of about 5, 10 or 15 mm). Diameter is here defined as the major dimension of an irregularly shaped agglomerate or the diameter of a spherical agglomerate.

Advantageously the treatment fluid is in an effective viscosity range for the fluid to at least partially permeate the agglomerates and coat at least a portion of the interior surface of the particles in the agglomerates prior to the bar becoming solidified. Preferably the fluid has a maximum viscosity of 1000 cps measured with a shear rate of 10⁻¹s using a Brookfield RVF viscometer and RV5 Spindle for 30 seconds taken at the agglomerate processing temperature that yields a viscosity within this range. More preferably the fluid has a minimum viscosity of about 0.7, 1, 2, 4, 10 or 15 cps and a maximum viscosity of 100, 200, 300, or 500 cps at 25 C; or where the viscosity is measured at the processing temperature of the bar. Advantageously the portion of the agglomerate's interior surface coated by the treatment fluid is greater than about 1, 2, 5, 10, 11, 15, 20, 30, 40, 50, 60, 70, 80 or 90 percent of the total interior surface area available for coating. Preferably the treatment fluid is selected from polyhydric alcohols (such as glycerin, propylene glycol and the like), fatty acids (such as stearic acid, coconut fatty acid, and the like); polyols (such as polyethylene glycol of about 200 to 10,000 in molecular weight, and the like) tri and diglyceride oils (such as plant derived, animal derived, or synthetic oils or blends and derivatives thereof), petroleum oils (such as molten petrolatum or wax, mineral oil and the like); silicone oils, fluorinated oils, brominated oils, C14 and lower alkyl esters; C14 and lower alkyl alcohols; or blends and derivatives thereof. Advantageously the treatment fluid has less than about 10% of water (preferably less than about 5, 3, or 1, and more preferably is anhydrous).

Advantageously the plurality of particles is agglomerated prior to being formulated into the bar, is agglomerated during bar formulation, or is agglomerated using a combination thereof. Preferably the plurality of particles is agglomerated prior to bar formulation.

Preferably the plurality of particles are selected from hydrophilic clays (such as Kaolin, bentonite etc.); hydrophobically modified clays (such as Kaolin, bentonite and the like); silica; zeolite; cellulose; starch (such as gelatinized starch, crosslinked starch, cyclodextrin starch, and the like) or blends and derivatives thereof. Most preferably the plurality of particles are selected from bentonite clay agglomerates, such as supplied by Bentonit Uniao Nordeste S. A. (Brazil), or cyclodextrin starch granulated by well known starch processing technology and supplied by such companies as Cerestar (Mechelen, Belgium)).

In one preferred embodiment of the massaging bar, the one or more anionic surfactants includes
a. about 0 to 30% by wt. of one or more fatty acid soaps; and
b. about 15 to 60% by wt. of one or more non-soap anionic surfactants. Preferably this bar contains about 0 to 20% by wt. of one or more fatty acid soaps and about 20 to 55% by wt. of one or more non-soap anionic surfactants.

In another preferred embodiment of the massaging bar, the one or more anionic surfactants includes
a. about 30 to 80% by wt. of one or more fatty acid soaps; and
b. about 5 to 40% by wt. of one or more non-soap anionic surfactants. Preferably this bar contains about 40 to 70% by wt. of one or more fatty acid soaps and about 7 to 30% by wt. of one or more non-soap anionic surfactants.

In another preferred embodiment of the massaging bar, the one or more anionic surfactants includes
a. about 30 to 80% by wt. of one or more fatty acid soaps; and
b. about 0 to 10% by wt. of one or more non-soap anionic surfactants. Preferably this bar contains about 50 to 80% by wt. of one or more fatty acid soaps and about 0 to 7% of one or more non-soap anionic surfactants.

The inventive massaging bar provides effective exfoliation (as measured by the exfoliation test described below) while reducing the sensation of skin abrasiveness (as measured by the skin abrasiveness test defined below) compared to comparative exfoliation bars. Furthermore the inventive bar maintains or improves skin smoothness and skin softness (as measured by the skin smoothness and softness tests defined below as compared to comparative exfoliation bars with less perception of exfoliation as measured by the bar sensory exfoliation index method as described below compared to comparative bars.

In another aspect of the invention is a process for manufacturing a massaging bar, including but not limited to the steps of in no particular order:
a. contacting particle agglomerates (preferably having a diameter in the range of about 0.1-20.0 mm) with a treatment fluid under conditions effective (preferably for a time in the range of about 10 seconds to 24 hours, agitation in a recirculation loop and temperature in the range of about 20 C to 30 C) for the agglomerate to have attained a fracture index of less than 1.0 to form a pretreated agglomerate, the fluid being selected from a hydrophilic compound, a hydrophobic compound or a blend thereof;
b. blending the pretreated agglomerate with at least one component of a toilet bar base selected from a soap base, a syndet base, or a combar base, optionally containing a fragrance and other minor components to form a mixed mass;
c. treating the mixed mass via an effective combination of milling, refining and/or plodding to form a refined mass; and
d. processing the refined mass by either extruding followed by stamping, cutting or a combination thereof; or casting followed by hardening to form a massaging bar.

In another aspect of the present inveniton is a method of skin cleansing with a massaging bar, including but not limited to the steps of:
a. wetting a massaging bar with water; the bar including:
   1. about 5 to 80% by wt. of one or more anionic surfactants;
   2. about 0.02 to 20% by wt. of water-disintegrable agglomerates, the agglomerates including a plurality of particles, optionally containing a binder; and a treatment fluid coating at least a portion of an interior surface of the particles in the agglomerates, the fluid being selected from a hydrophilic compound, a hydrophobic compound or a blend thereof;
   3. wherein the agglomerates have a fracture index less than 1.0; and
   4. wherein the bar has a rigidity index of greater than 0.2 Mpa at 25 C; and
b. rubbing the wet bar on the skin with sufficient force to cause the agglomerates to disintegrate while cleansing and massaging the skin.

Surfactants:

Surfactants are an essential component of the inventive toilet bar. They are compounds that have hydrophobic and hydrophilic portions that act to reduce the surface tension of the aqueous solutions they are dissolved in. Useful surfactants can include anionic, nonionic, amphoteric, and cationic surfactants, and blends thereof.

Anionic Surfactants:

Synthetic Anionic Surfactants

The massaging bar of the present invention may contain one or more non-soap anionic detergents (syndets). Preferably the syndets have a zein value of 50 or less. Zein value may be measured using the test method described below.

The anionic detergent active which may be used may be aliphatic sulfonates, such as a primary alkane (e.g., C₈-C₂₂) sulfonate, primary alkane (e.g., C₈-C₂₂) disulfonate, C₈-C₂₂ alkene sulfonate, C₈-C₂₂ hydroxyalkane sulfonate or alkyl glyceryl ether sulfonate (AGS); or aromatic sulfonates such as alkyl benzene sulfonate.

The anionic may also be an alkyl sulfate (e.g., C₁₂-C₁₈ alkyl sulfate) or alkyl ether sulfate (including alkyl glyceryl ether sulfates). Among the alkyl ether sulfates are those having the formula:
RO(CH₂CH₂O)ₙSO₃M
wherein R is an alkyl or alkenyl having 8 to 18 carbons, preferably 12 to 18 carbons, n has an average value of greater than 1.0, preferably greater than 3; and M is a solubilizing cation such as sodium, potassium, ammonium or substituted ammonium. Ammonium and sodium lauryl ether sulfates are preferred.

The anionic may also be alkyl sulfosuccinates (including mono- and dialkyl, e.g., C₆-C₂₂ sulfosuccinates); alkyl and acyl taurates, alkyl and acyl sarcosinates, sulfoacetates, C₈-C₂₂ alkyl phosphates and phosphates, alkyl phosphate esters and alkoxyl alkyl phosphate esters, acyl lactates, C₈-C₂₂ monoalkyl succinates and maleates, sulphoacetates, alkyl glucosides and acyl isethionates, and the like.

Sulfosuccinates may be monoalkyl sulfosuccinates having the formula:
R⁴O₂CCH₂CH(SO₃M)CO₂M; and
amide-MEA sulfosuccinates of the formula;
R⁴CONHCH₂CH₂O₂CCH₂CH(SO₃M)CO₂M
wherein R⁴ ranges from C₈-C₂₂ alkyl and M is a solubilizing cation.

Sarcosinates are generally indicated by the formula:
R¹CON(CH₃)CH₂CO₂M,
wherein R¹ ranges from C₈-C₂₀ alkyl and M is a solubilizing cation.

Taurates are generally identified by formula:
R²CONR³CH₂CH₂SO₃M
wherein R² ranges from C₈-C₂₀ alkyl, R³ ranges from C₁-C₄ alkyl and M is a solubilizing cation.

The inventive massaging bar preferably contains C₈-C₁₄ acyl isethionates. These esters are prepared by reaction between alkali metal isethionate with mixed aliphatic fatty acids having from 6 to 12 carbon atoms and an iodine value of less than 20.

The acyl isethionate may be an alkoxylated isethionate such as is described in Ilardi et al., U.S. Pat. No. 5,393,466, titled “Fatty Acid Esters of Polyalkoxylated isethonic acid; issued Feb. 28, 1995; hereby incorporated by reference. This compound has the general formula: 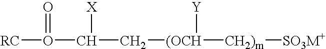
wherein R is an alkyl group having 8 to 18 carbons, m is an integer from 1 to 4, X and Y are hydrogen or an alkyl group having 1 to 4 carbons and M⁺ is a monovalent cation such as, for example, sodium, potassium or ammonium.

In another embodiment of the inventive toilet bar, there is less than 5% by wt. of any of the following anionic surfactants: alkyl sulfates, alkyl sulfonates, alkyl benzene sulfonates, alkyl alkoxy sulfates, acyl taurides, acyl sulfates, and polyhydroxy fatty acid amides either individually or of a blend thereof. Preferably there is less than 1%, and more preferably less than 0.1% by wt. of these surfactants

Soaps.

The inventive massaging bar may contain soap. The term “soap” is used herein in its popular sense, i.e., the alkali metal or alkanol ammonium salts of alkane- or alkene monocarboxylic acids. Sodium, potassium, mono-, di- and tri-ethanol ammonium cations, or combinations thereof, are suitable for purposes of this invention. In general, sodium, potassium, ammonium, mono-, di-, and tri-ethanol soaps of saturated C8-C14 alkyl chains and unsaturated fatty acids, preferably having C8-C22 alkyl chains, are soluble soaps.

Amphoteric Surfactants

One or more amphoteric surfactants may be used in this invention. Such surfactants include at least one acid group. This may be a carboxylic or a sulphonic acid group. They include quaternary nitrogen and therefore are quaternary amido acids. They should generally include an alkyl or alkenyl group of 7 to 18 carbon atoms. They will usually comply with an overall structural formula: 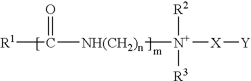
where R¹ is alkyl or alkenyl of 7 to 18 carbon atoms;
R² and R³ are each independently alkyl, hydroxyalkyl or carboxyalkyl of 1 to 3 carbon atoms;
n is 2 to 4;
m is 0 to 1;
X is alkylene of 1 to 3 carbon atoms optionally substituted with hydroxyl, and
Y is —CO₂— or —SO₃—

Suitable amphoteric surfactants within the above general formula include simple betaines of formula: 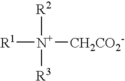
and amido betaines of formula: 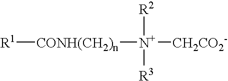
where n is 2 or 3.

In both formulae R¹, R² and R³ are as defined previously. R¹ may in particular be a mixture of C₁₂ and C₁₄ alkyl groups derived from coconut oil so that at least half, preferably at least three quarters of the groups R¹ have 10 to 14 carbon atoms. R² and R³ are preferably methyl.

A further possibility is that the amphoteric detergent is a sulphobetaine of formula: 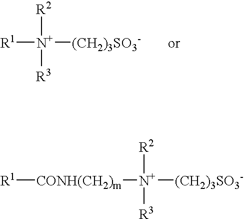
where m is 2 or 3, or variants of these in which —(CH₂)₃ SO₃⁻ is replaced by

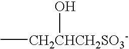

In these formulae R¹, R² and R³ are as discussed previously.

Amphoacetates and diamphoacetates are also intended to be covered in possible zwitterionic and/or amphoteric compounds which may be used such as e.g., sodium lauroamphoacetate, sodium cocoamphoacetate, and blends thereof, and the like.

Nonionic Surfactants

One or more nonionic surfactants may also be used in the massaging bar of the present invention.

The nonionics which may be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic detergent compounds are alkyl (C₆-C₂₂) phenols ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other so-called nonionic detergent compounds include long chain tertiary amine oxides, long chain tertiary phosphine oxides and dialkyl sulphoxide, and the like.

The nonionic may also be a sugar amide, such as a polysaccharide amide. Specifically, the surfactant may be one of the lactobionamides described in U.S. Pat. No. 5,389,279 to Au et al. titled “Compositions Comprising Nonionic Glycolipid Surfactants issued Feb. 14, 1995; which is hereby incorporated by reference or it may be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference.

Cationic Skin Conditioning Agents

An optional component in compositions according to the invention is a cationic skin feel agent or polymer, such as for example cationic celluloses. Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200, and quaternary ammonium compounds such as alkyldimethylammonium halogen ides.

A particularly suitable type of cationic polysaccharide polymer that can be used is a cationic guar gum derivative, such as guar hydroxypropyltrimonium chloride (Commercially available from Rhone-Poulenc in their JAGUAR trademark series). Examples are JAGUAR C13S, which has a low degree of substitution of the cationic groups and high viscosity, JAGUAR C15, having a moderate degree of substitution and a low viscosity, JAGUAR C17 (high degree of substitution, high viscosity), JAGUAR C16, which is a hydroxypropylated cationic guar derivative containing a low level of substituent groups as well as cationic quaternary ammonium groups, and JAGUAR 162 which is a high transparency, medium viscosity guar having a low degree of substitution.

Particularly preferred cationic polymers are JAGUAR C13S, JAGUAR C15, JAGUAR C17 and JAGUAR C16 and JAGUAR C162, especially Jaguar C13S. Other cationic skin feel agents known in the art may be used provided that they are compatible with the inventive formulation.

Other preferred cationic compounds that are useful in the present invention include amido quaternary ammonium compounds such as quaternary ammonium propionate and lactate salts, and quaternary ammonium hydrolyzates of silk or wheat protein, and the like. Many of these compounds can be obtained as the Mackine™ Amido Functional Amines, Mackalene™ Amido functional Tertiary Amine Salts, and Mackpro® cationic protein hydrolysates from the McIntyre Group Ltd. (University Park, Ill.).

In a preferred embodiment of the invention having a hydrolyzed protein conditioning agent, the average molecular weight of the hydrolyzed protein is preferably about 2500. Preferably 90% of the hydrolyzed protein is between a molecular weight of about 1500 to about 3500. In a preferred embodiment, MACKPRO™ WWP (i.e. wheat germ amido dimethylamine hydrolyzed wheat protein) is added at a concentration of 0.1% (as is) in the bar. This results in a MACKPRO™ WWP “solids” of 0.035% in the final bar formula for this embodiment.

Cationic Surfactants

One or more cationic surfactants may also be used in the inventive massaging bar.

Examples of cationic detergents are the quaternary ammonium compounds such as alkyldimethylammonium halogenides.

Other suitable surfactants which may be used are described in U.S. Pat. No. 3,723,325 to Parran Jr. titled “Detergent Compositions Containing Particle Deposition Enhancing Agents” issued Mar. 27, 1973; and “Surface Active Agents and Detergents” (Vol. I & II) by Schwartz, Perry & Berch, both of which are also incorporated into the subject application by reference.

In addition, the inventive composition, especially the toilet bar of the invention may include 0 to 15% by wt. optional ingredients as follows:
perfumes; sequestering agents, such as tetrasodium ethylenediaminetetraacetate (EDTA), EHDP or mixtures in an amount of 0.01 to 1%, preferably 0.01 to 0.05%; and coloring agents, opacifiers and pearlizers such as zinc stearate, magnesium stearate, TiO₂, EGMS (ethylene glycol monostearate) or Lytron 621 (Styrene/Acrylate copolymer) and the like; all of which are useful in enhancing the appearance or cosmetic properties of the product.

The compositions may further comprise preservatives such as dimethyloldimethylhydantoin (Glydant XL1000), parabens, sorbic acid etc., and the like.

The compositions may also comprise coconut acyl mono- or diethanol amides as suds boosters, and strongly ionizing salts such as sodium chloride and sodium sulfate may also be used to advantage.

Antioxidants such as, for example, butylated hydroxytoluene (BHT) and the like may be used advantageously in amounts of about 0.01% or higher if appropriate.

In addition to the agglomerates, moisturizers also expressed as either humectants and emollients may be advantageously used in the present invention. Humectants such as polyhydric alcohols, e.g. glycerin and propylene glycol, and the like; and polyols such as the polyethylene glycols listed below and the like may be used. Humectants may be employed at levels greater than 0.01, 0.05, 0.1, 0.2, 0.5, 0.9, 1.0, 1.1, 2.0, 3.0, 5, 9, 10, 11, 15 or 20% by wt.

| | |
|---|---|
| Polyox WSR-205 | PEG 14M, |
| Polyox WSR-N-60K | PEG 45M, or |
| Polyox WSR-N-750 | PEG 7M. |

Emollients may be added to the inventive product separately from agglomerates or at least a portion of agglomerates may be used that has been enriched in emollients. These separate emollients that are distinct from the agglomerates may be composed of a single moisturizing agent component or they may be a mixture of two or more compounds one or all of which may have a moisturizing aspect. In addition, the moisturizing agent itself may act as a carrier for other components one may wish to add to the inventive massaging bar.

Hydrophobic emollients, hydrophilic emollients, or a blend thereof may be used. Preferably, hydrophobic emollients are used in excess of hydrophilic emollients in the inventive toilet skin care or cleansing composition. Hydrophobic emollients are preferably present in a concentration greater than about 0.01, 0.05, 0.1, 0.2, 0.5, 0.9, 1.0, 1.1, 2.0, 3.0, 5, 9, 10, 11, 15, 20, 25 or 30% by wt. Hydrophobic emollients added separately from the agglomerates may be present in the inventive product at levels greater than about 0.01, 0.05, 0.1, 0.2, 0.5, 0.9, 1.0, 1.1, 2.0, 3.0, 5, 9, 10, 11, 15, or 20% by wt. The term “emollient” is defined as a substance which softens or improves the elasticity, appearance, and youthfulness of the skin (stratum corneum) by increasing its water content, and keeps it soft by retarding the decrease of its water content.

Useful emollients include the following:
(a) silicone oils and modifications thereof such as linear and cyclic polydimethylsiloxanes; amino, alkyl, alkylaryl, and aryl silicone oils;
(b) fats and oils including natural fats and oils such as jojoba, soybean, sunflower, rice bran, avocado, almond, olive, sesame, persic, castor, coconut, mink oils; cacao fat; beef tallow, lard; hardened oils obtained by hydrogenating the aforementioned oils; and synthetic mono, di and triglycerides such as myristic acid glyceride and 2-ethylhexanoic acid glyceride;
(c) waxes such as carnauba, spermaceti, beeswax, lanolin, and derivatives thereof;
(d) hydrophobic and hydrophilic plant extracts;
(e) hydrocarbons such as liquid paraffin, vaseline, microcrystalline wax, ceresin, squalene, pristan and mineral oil;
(f) higher fatty acids such as lauric, myristic, palmitic, stearic, behenic, oleic, linoleic, linolenic, lanolic, isostearic, arachidonic and poly unsaturated fatty acids (PUFA);
(g) higher alcohols such as lauryl, cetyl, stearyl, oleyl, behenyl, cholesterol and 2-hexydecanol alcohol;
(h) esters such as cetyl octanoate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate;
(i) essential oils and extracts thereof such as mentha, jasmine, camphor, white cedar, bitter orange peel, ryu, turpentine, cinnamon, bergamot, citrus unshiu, calamus, pine, lavender, bay, clove, hiba, eucalyptus, lemon, starflower, thyme, peppermint, rose, sage, sesame, ginger, basil, juniper, lemon grass, rosemary, rosewood, avocado, grape, grapeseed, myrrh, cucumber, watercress, calendula, elder flower, geranium, linden blossom, amaranth, seaweed, ginko, ginseng, carrot, guarana, tea tree, jojoba, comfrey, oatmeal, cocoa, neroli, vanilla, green tea, penny royal, aloe vera, menthol, cineole, eugenol, citral, citronelle, borneol, linalool, geraniol, evening primrose, camphor, thymol, spirantol, penene, limonene and terpenoid oils; and
(j) mixtures of any of the foregoing components, and the like.

Preferred emollient moisturizing agents are selected from fatty acids, triglyceride oils, mineral oils, petrolatum, and mixtures thereof. Further preferred emollients are fatty acids.

Exfoliants

Additional exfoliants other than the agglomerates may be used. Such exfoliants used in the art include natural minerals such as silica, talc, calcite, pumice, tricalcium phosphate; seeds such as rice, apricot seeds, etc; crushed shells such as almond and walnut shells; oatmeal; polymers such as polyethylene and polypropylene beads, flower petals and leaves; microcrystalline wax beads; jojoba ester beads, and the like. These exfoliants come in a variety of particle sizes and morphology ranging from micron sized to a few mm. They also have a range of hardness. Some examples are given in table 1 below. Preferably such additional exfoliants are selected from softer varieties such as those with a Mohs hardenss of less than about 4, 3, or 2. In a preferred embodiment, no additional exfoliants are used.

TABLE 1

| Material | Hardness (Mohs) |
|---|---|
| Talc | 1 |
| Calcite | 3 |
| Pumice | 4-6 |
| Walnut Shells | 3-4 |
| Dolomite | 4 |
| Polyethylene | ˜1 |

Optional Active Agents

Advantageously, active agents other than moisturizers defined above may be added to the agglomerate in a safe and effective amount before or during formulation of the inventive massaging bar in order to immobilize and/or stabilize such ingredients so as to allow more controlled release and effective utilization of these ingredients to and by the skin during the use of the product. These active ingredients may be advantageously selected from antimicrobial and antifungal actives, vitamins, anti-acne actives; anti-wrinkle, anti-skin atrophy and skin repair actives; skin barrier repair actives; non- steroidal cosmetic soothing actives; artificial tanning agents and accelerators; skin lightening actives; sunscreen actives; sebum stimulators; sebum inhibitors; anti-oxidants; protease inhibitors; skin tightening agents; anti-itch ingredients; hair growth inhibitors; 5-alpha reductase inhibitors; desquamating enzyme enhancers; anti-glycation agents; topical anesthetics, or mixtures thereof; and the like.

These active agents may be selected from water soluble active agents, oil soluble active agents, pharmaceutically-acceptable salts and mixtures thereof. Advantageously the agents will be soluble or dispersible in the treatment fluid used to treat the agglomerate. The term “active agent” as used herein, means personal care actives which can be used to deliver a benefit to the skin and/or hair and which generally are not used to confer a conditioning benefit, as is conferred by humectants and emollients previously described herein. The term “safe and effective amount” as used herein, means an amount of active agent high enough to modify the condition to be treated or to deliver the desired skin care benefit, but low enough to avoid serious side effects. The term “benefit,” as used herein, means the therapeutic, prophylactic, and/or chronic benefits associated with treating a particular condition with one or more of the active agents described herein. What is a safe and effective amount of the active agent ingredient will vary with the specific active agent, the ability of the active to penetrate through the skin, the age, health condition, and skin condition of the user, and other like factors. Preferably the massaging bars of the present invention comprise from about 0.01% to about 50%, more preferably from about 0.05% to about 25%, even more preferably 0.1% to about 10%, and most preferably 0.1% % to about 5%, by weight of the active agent component.

Anti-acne actives can be effective in treating acne vulgaris, a chronic disorder of the pilosebaceous follicles. Nonlimiting examples of useful anti-acne actives include the keratolytics such as salicylic acid (o-hydroxybenzoic acid), derivatives of salicylic acid such as 5-octanoyl salicylic acid and 4 methoxysalicylic acid, and resorcinol; retinoids such as retinoic acid and its derivatives (e.g., cis and trans); sulfur-containing D and L amino acids and their derivatives and salts, particularly their N-acetyl derivatives, mixtures thereof and the like.

Antimicrobial and antifungal actives can be effective to prevent the proliferation and growth of bacteria and fungi. Nonlimiting examples of antimicrobial and antifungal actives include b-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, triclosan; triclocarban; and mixtures thereof and the like.

Anti-wrinkle, anti-skin atrophy and skin repair actives can be effective in replenishing or rejuvenating the epidermal layer. These actives generally provide these desirable skin care benefits by promoting or maintaining the natural process of desquamation. Nonlimiting examples of antiwrinkle and anti-skin atrophy actives include vitamins, minerals, and skin nutrients such as milk, vitamins A, E, and K; vitamin alkyl esters, including vitamin C alkyl esters; magnesium, calcium, copper, zinc and other metallic components; retinoic acid and its derivatives (e.g., cis and trans); retinal; retinol; retinyl esters such as retinyl acetate, retinyl palmitate, and retinyl propionate; vitamin B 3 compounds (such as niacinamide and nicotinic acid), alpha hydroxy acids, beta hydroxy acids, e.g. salicylic acid and derivatives thereof (such as 5-octanoyl salicylic acid, heptyloxy 4 salicylic acid, and 4-methoxy salicylic acid); mixtures thereof and the like.

Skin barrier repair actives are those skin care actives which can help repair and replenish the natural moisture barrier function of the epidermis. Nonlimiting examples of skin barrier repair actives include lipids such as cholesterol, ceramides, sucrose esters and pseudo-ceramides as described in European Patent Specification No. 556,957; ascorbic acid; biotin; biotin esters; phospholipids, mixtures thereof, and the like.

Non-steroidal Cosmetic Soothing Actives can be effective in preventing or treating inflammation of the skin. The soothing active enhances the skin appearance benefits of the present invention, e.g., such agents contribute to a more uniform and acceptable skin tone or color. Nonlimiting examples of cosmetic soothing agents include the following categories: propionic acid derivatives; acetic acid derivatives; fenamic acid derivatives; mixtures thereof and the like. Many of these cosmetic soothing actives are described in U.S. Pat. No. 4,985,459 to Sunshine et al., issued Jan. 15, 1991, incorporated by reference herein in its entirety.

Artificial tanning actives can help in simulating a natural suntan by increasing melanin in the skin or by producing the appearance of increased melanin in the skin. Nonlimiting examples of artificial tanning agents and accelerators include dihydroxyacetaone; tyrosine; tyrosine esters such as ethyl tyrosinate and glucose tyrosinate; mixtures thereof, and the like.

Skin lightening actives can actually decrease the amount of melanin in the skin or provide such an effect by other mechanisms. Nonlimiting examples of skin lightening actives useful herein include aloe extract, alpha-glyceryl-L-ascorbic acid, aminotyroxine, ammonium lactate, glycolic acid, hydroquinone, 4 hydroxyanisole, mixtures thereof, and the like.

Also useful herein are sunscreen actives. A wide variety of sunscreen agents are described in U.S. Pat. No. 5,087,445, to Haffey et al., issued Feb. 11, 1992; U.S. Pat. No. 5,073,372, to Turner et al., issued Dec. 17, 1991; U.S. Pat. No. 5,073,371, to Turner et al. issued Dec. 17, 1991; and Segarin, et al., at Chapter VIII, pages 189 et seq., of Cosmetics Science and Technology, all of which are incorporated herein by reference in their entirety. Nonlimiting examples of sunscreens which are useful in the compositions of the present invention are those selected from the group consisting of octyl methoxyl cinnamate (Parsol MCX) and butyl methoxy benzoylmethane (Parsol 1789), 2-ethylhexyl p-methoxycinnamate, 2-ethylhexyl N,N-dimethyl-p-aminobenzoate, p-aminobenzoic acid, 2-phenylbenzimidazole-5-sulfonic acid, oxybenzone, mixtures thereof, and the like.

Sebum stimulators can increase the production of sebum by the sebaceous glands. Nonlimiting examples of sebum stimulating actives include bryonolic acid, dehydroetiandrosterone (DHEA), orizanol, mixtures thereof, and the like.

Sebum inhibitors can decrease the production of sebum by the sebaceous glands. Nonlimiting examples of useful sebum inhibiting actives include aluminum hydroxy chloride, corticosteroids, dehydroacetic acid and its salts, dichlorophenyl imidazoldioxolan (available from Elubiol), mixtures thereof, and the like.

Also useful as actives in the present invention are protease inhibitors. Protease inhibitors can be divided into two general classes: the proteinases and the peptidases. Proteinases act on specific interior peptide bonds of proteins and peptidases act on peptide bonds adjacent to a free amino or carboxyl group on the end of a protein and thus cleave the protein from the outside. The protease inhibitors suitable for use in the present invention include, but are not limited to, proteinases such as serine proteases, metalloproteases, cysteine proteases, and aspartyl protease, and peptidases, such as carboxypepidases, dipeptidases and aminopepidases, mixtures thereof and the like.

Other useful as active ingredients in the present invention are skin tightening agents. Nonlimiting examples of skin tightening agents which are useful in the compositions of the present invention include monomers which can bind a polymer to the skin such as terpolymers of vinylpyrrolidone, (meth)acrylic acid and a hydrophobic monomer comprised of long chain alkyl (meth)acrylates, mixtures thereof, and the like.

Active ingredients in the present invention may also include anti-itch ingredients. Suitable examples of anti-itch ingredients which are useful in the compositions of the present invention include hydrocortisone, methdilizine and trimeprazineare, mixtures thereof, and the like.

Nonlimiting examples of hair growth inhibitors which are useful in the compositions of the present invention include 17 beta estradiol, anti angiogenic steroids, curcuma extract, cycloxygenase inhibitors, evening primrose oil, linoleic acid and the like. Suitable 5-alpha reductase inhibitors such as ethynylestradiol and, genistine mixtures thereof, and the like.

Nonlimiting examples of desquamating enzyme enhancers which are useful in the compositions of the present invention include alanine, aspartic acid, N methyl serine, serine, trimethyl glycine, mixtures thereof, and the like.

A nonlimiting example of an anti-glycation agent which is useful in the compositions of the present invention would be Amadorine (available from Barnet Products Distributor), and the like.

Active agents may be immobilized within or adjacent to agglomerates by any art recognized technique such as by contacting the agglomerates with a solution or suspension (i.e. a transfer medium) of one or more active agents under conditions sufficient to allow the transfer of a desired quantity of active agent to the agglomerates contained in the massaging bar. Preferably the active agents may be added to the treatment fluid for the agglomerates. This step may then followed by the isolation of the agglomerates via filtration, evaporation of the solvent or any other art recognized process step to yield agglomerates containing active agent. Alternatively the agglomerates may be directly formulated the massaging bar without being isolated from the transfer medium provided the medium is compatible with the bar formulation to which it is added, or a combination of agglomerates and transfer medium may be used. The transfer medium may be a liquid, gel, solid, particulate or blend thereof, and the like.

Except in the operating and comparative examples, or where otherwise explicitly indicated, all numbers in this description indicating amounts of material ought to be understood as modified by the word “about”.

The following examples will more fully illustrate the embodiments of this invention. All parts, percentages and proportions referred to herein and in the appended claims are by weight unless otherwise illustrated. Physical test methods are described below:

The following inventive toilet bars may be formulated according to the manufacturing methods described below:

### EXAMPLE 1

### Normal Toilet Soaps

The pre-wet agglomerates are added in chip mixer along with soap base, fragrance and other minor components (where all concentrations are based on the finished bar). Gelatinized starch was produced by gelatinization of maze starch at 90 C in presence of water using art recognized techniques. This gelatinized starch slurry was dried and crushed to the desired particle size. The gelatinized starch granules are soaked in the below mentioned benefit and conditioning components to reduce the harsh feel of the particles on the skin.

The soap base can be of varying ratios of the different fatty acid soap components (one example is described below). The minor components can be the common ingredients used in soap bar manufacture including emollients, active agents, colorants, opacifiers, brightners etc. The mixed mass is then milled/refined and plodded. The plodded bars are pressed into the desired shape. Four examples of such bars (a, b, c and d) may be made as follows (concentrations are in % by wt. of the bar):

Bars

| Ingredients | a | b | c | d |
|---|---|---|---|---|
| Pre-wet agglomerates: | | | | |
| Sunflower Seed Oil | 1.0 | 0.5 | — | 0.5 |
| Mineral oil | — | — | 0.5 | — |
| Glycerin | — | — | — | 1.0 |
| Gelatinized Starch | 0.5 | 0.5 | — | — |
| Bentonite Granules | — | — | 0.5 | 0.5 |
| Other bar components: | | | | |
| Soap Base | 87.0 | 87.3 | 87.5 | 86.5 |
| Perfume | 1.0 | 1.0 | 1.0 | 1.0 |
| Irgason DP 300 | — | 0.2 | — | — |
| Titanium Dioxide | 0.5 | 0.5 | 0.5 | 0.5 |
| Water | 10.0 | 10.0 | 10.0 | 10.0 |
| Total | 100 | 100 | 100 | 100 |

### EXAMPLE 2

### Massaging Combar

The pre-wet granules can be added in chip mixer along with the combar base (described below), having a mixture of soapy and non-soapy actives and other minor components. The mixed mass is then milled/refined and plodded. The plodded bars are pressed into the desired shape. Three examples of such bars e, f, and g may be made as follows (concentrations are in % by wt. of the bar):

| Ingredients | e | f | g |
|---|---|---|---|
| Pre-wet agglomerates: | | | |
| Gelatinized Starch | 0.5 | 0.5 | — |
| Bentonite Granules | — | — | 0.5 |
| Sunflower Seed Oil | 1.0 | — | — |
| Mineral Oil | — | 1.0 | — |
| Propylene Glycol | — | — | 1.0 |
| Other bar components: | | | |
| Soap Base | 76.8 | 83.8 | 71.5 |
| Sodium Cocoyl Isethionate | 10.0 | | 10.0 |
| Alfa Olefin Sulfonate | | 3.0 | 3.0 |
| Cocoamido Propyl Betaine | | | 2.0 |
| Perfume | 1.0 | 1.0 | 1.0 |
| Salt (sodium chloride) | 0.7 | 0.7 | 1.0 |
| Water | 10.0 | 10.0 | 10.0 |
| Total | 100 | 100 | 100 |

### EXAMPLE 2A

### Massaging Combar

A further example of an inventive toilet bar q′ prepared according to the method of Example 2 was made as follows:

| Ingredient | % | % Active |
|---|---|---|
| 70/30 Tallow/Coco Soap Noodles | 72.66 | 68 |
| EHDP | 0.02 | 60 |
| EDTA | 0.02 | 39 |
| Sulfomethylester | 2 | 44.5 |
| Betaine | 1.8 | 28.2 |
| Stearic Acid | 5 | 100 |
| Glycerin | 0.5 | 100 |
| Water | 11.5 | 100 |
| Merquat 100 or Mackernium | 0.2 | 40 |
| Coconut Fatty Acid | 0.5 | 100 |
| Titanium Dioxide | 0.6 | 100 |
| Mineral Oil (Heavy) - Hydrobrite 1000 | 0.5 | 100 |
| Petrolatum - (2.5 Hard) | 1 | 100 |
| Softben-10 (0.3-1.0 mm) | 1 | 100 |
| Softben-10 (1.4-2.0 mm) | 1 | 100 |
| A-C Polyethylene 9A | 0.05 | 100 |
| Sunflower Seed Oil | 0.5 | 100 |
| Fragrance | 1.05 | 100 |
| Wheat Protein - MacPro WWP | 0.1 | 35 |
| Total | 100 | |

Note:
Sulfomethylester known as Alpha Step BSS-45 from Stepan

### EXAMPLE 3

### Massaging Syndet Bar

The pre-wet agglomerated particles of the massaging components can be added in chip mixer along with syndet base (described below), having a mixture of non-soapy actives and the other minor components. A small quantity of soap base can be added in chip mixer to facilitate the process. The mixed mass is then milled/refined and plodded. The plodded bars are pressed into the desired shape. The base is made by melting sodium cocoyl isethionate with stearic acid, salt and small quantity of soap. This molten mass is chill roll milled and then used as base. Three examples of such bars (h, i and j) may be made as follows (concentrations are in % by wt. of the bar):

| Ingredients | h | i | j |
|---|---|---|---|
| Pre-wet agglomerates: | | | |
| Gelatinized Starch | 0.5 | 0.5 | — |
| Bentonite Granules | — | — | 0.5 |
| Sunflower Seed Oil | — | — | 3.0 |
| Mineral Oil | 1.0 | — | — |
| Glycerin | — | 0.5 | — |
| Other bar components: | | | |
| Syndet Soap Base | 92.5 | 93.0 | 90.5 |
| Perfume | 1.0 | 1.0 | 1.0 |
| Water | 5.0 | 5.0 | 5.0 |
| Total | 100 | 100 | 100 |

### EXAMPLE 4

### Translucent Bar

First translucency is created in the transparent soap base (described below), by high shear mixing at 47-50 C for about 30 minutes to 50 minutes. The pre-treated agglomerates are added in the mixer along with fragrance at the end of this 30 to 50 minute period and blended for about 5 minutes. The mass is then plodded under controlled conditions of temperature to get a translucent bar with the dispersed agglomerates. Four examples of such bars (k, l, m and n) may be made as follows (concentrations are in % by wt. of the bar):

| Ingredients | k | l | m | n |
|---|---|---|---|---|
| Pre-wet agglomerates: | | | | |
| Gelatinized Starch | 0.15 | 0.15 | — | |
| Bentonite Granules | — | — | 0.15 | 0.15 |
| Sunflower Seed Oil | 0.35 | — | — | |
| Mineral Oil | — | 0.35 | — | |
| Propylene Glycol | — | — | 0.35 | 0.35 |
| Other bar components: | | | | |
| 82/18 Soap¹ (anhydrous) | 69.5 | 71.5 | 72.5 | 72.5 |
| Glycerin | 5.0 | 3.0 | 5.0 | — |
| Propylene Glycol | 5.0 | 3.0 | — | 5.0 |
| Triethanolamine | — | 3.0 | 3.0 | 3.0 |
| Perfume | 1.0 | 1.0 | 1.0 | 1.0 |
| Water | 18.0 | 18.0 | 18.0 | 18.0 |
| Total | 100 | 100 | 100 | 100 |

¹Tallowate/Cocoate

### EXAMPLE 5

### Melt Cast Bar Formulations

Melt cast bars may also be made depending on the melt properties of the particular blend used. In this case, the formulation is made by melting all the ingredients at 60-95° C. The homogeneous liquid, whether transparent or opaque, is cooled to preferably at 65-70°. The pre-treated agglomerates and fragrance are added at this temperature in the melt, homogenized and then product is poured into molds. The blend is then allowed to solidify under ambient or accelerated cooling conditions. It is preferred to premix the agglomerates in glycerin or any other suitable liquid for better dispersion. The bars after cooling can be transparent, translucent or opaque depending upon the formulation. Use of hydrophobic moisturizing components such as triglycerides in transparent soap formulations may reduce the transparency. The agglomerates advantageously holds the oil in it, thereby preserving the transparency and providing an attractive visual effect in the bar. Control of particle size and the viscosity of the melt are critical for stable suspension of the powder. Three examples of opaque melt cast formulations (o, p and q) are as follows (concentrations are in % by wt. of the bar):

| Ingredients | o | p | q |
|---|---|---|---|
| Pre-wet agglomerates: | | | |
| Gelatinized Starch | 1.5 | 2.5 | — |
| Bentonite Granules | — | — | 1.5 |
| Sunflower Seed Oil | 3.5 | — | — |
| Mineral Oil | — | 4.5 | — |
| Propylene Glycol | — | — | 3.5 |
| Other bar components: | | | |
| Sodium Cocoyl Isethionate | 40.77 | 42.6 | 42.00 |
| Stearic acid/Palmitic Acid | 10.00 | 10.00 | 10.00 |
| Coco Fatty acid | 0.67 | — | — |
| Sodium Isethionate | 0.56 | — | — |
| Alfa Olefin Sulfonate | 5.00 | 3.70 | 4.00 |
| Sodium Lauryl Ether | 5.00 | 3.7 | 1.00 |
| Sulfate (2 EO) | | | |
| Perfume | 1.00 | 1.00 | 1.00 |
| Titanium Dioxide | 1.00 | 1.00 | 1.00 |
| Propylene Glycol | 7.00 | 5.00 | 5.00 |
| Glycerin | 4.00 | 4.00 | 6.00 |
| Lauryl Alcohol | 5.00 | 5.00 | 6.00 |
| 12-hydroxystearic acid | 11.00 | 13.00 | 15.00 |
| Water | q.s. to 100% | q.s. to 100% | q.s. to 100% |

TABLE 2
Toilet bar bases:

| | |
|---|---|
| Soap Base | Syndet Base |

| | | | |
|---|---|---|---|
| 50-90% | Tallow Soap | 40-60% | SCI |
| 10-50% | Coco Soap | 2-8% | Alfa Olefin Sulfonate |
| 200-500 | ppm EDTA | 15-40% | Stearic Acid |
| 0.1-1.5% | Salt | 0-15% | Tallowate/Cocoate Soap |

### DESCRIPTION OF TEST METHODS

Methods of Testing

One or more of the following tests can be used to characterize the massaging toilet bars and compare it to comparative toilet bars.

a. Fracture Index

An amount of agglomerate is weighed and placed on a test stand. An Instron Model-4500 instrument using a compression program running at 1 mm/minute is used to apply an increasing load until the agglomerate fractures. This reading is compared to the fracture load obtained for an equal amount of agglomerate that has been pretreated with a treatment fluid. The fracture load measured for the treated agglomerate divided by the load measured for the same agglomerate without treatment is the Fracture index.

b. Rigidity Index.

Equipment used: Instron

Basic Method:

1. Load Cell with the solid/semi-solid bar that is equilibrated and maintained at a constant temperature of 25 C using a water bath.

2. Apply force from the top so that the solid/semi-solid bar is displaced at a rate of 10 mm/min through an orifice at the bottom of cell and measure the force applied in kN. The orifice has a diameter of 2 mm and a length of 60 mm.

The Pressure drop across the capillary length defines how rigid the material is. This rigidity index is defined as:
Rigidity Index=Pressure drop (kPA)=Force (kN)/Area (m\hat{ }2)
Where the Area=PI*Diam\hat{ }2/4
Diam=diameter of the rod applying the force, in this case 31.4 mm.

c. Exfoliation Test:

A suitable corneocyte staining dye (e.g.: gentian violet) is applied to a 2-5 cm diameter spot on skin (arm/leg or any other part of the body as desired) and left on for 5 minutes to ensure uniform staining of the skin surface cells (corneocytes). The excess dye is then washed away by rinsing the spot under running water at 35 C for 30 sec. with no rubbing of the skin.

The stained sites are then washed with the test product. For the bar the following wash method is adopted. Wet the spot on skin, pre-wet the bar, rub bar directly on spot for 30 sec (back and forth motion), rinse for 15 sec under running water at 35 C for 30 sec. with no rubbing of the skin and gently pat dry. Allow the site to dry for 10 minutes. A d-squame tape (Cuderm® manufactured by CuDerm Corporation, (Dallas, Tex.) is applied on the washed spot under a uniform pressure for 30 sec. and then removed. The d-squame tape is imaged using a Kodak DCS 420 digital camera with a 105 mm lens. The image is analyzed using Optimas image analysis software for area covered/total intensity of stained cells (Optimas® is available from Media Cybernetics, Silver Springs, Md.). By comparing this data to similar information from an unwashed site, one can estimate the amount of exfoliation caused by the test product as follows:
Exfoliation=(area of d-squame covered by stain on unwashed site−area of d-squame covered by stain on washed site)/(area of d-squame covered by stain on unwashed site)

Alternately exfoliation can also be evaluated in a consumer test as follows:

The test protocol consists of
1) Recruiting approx. 10-20 women in the age group of 25-65 and who are complexion bar users.
2) Use test and comparative products for a week each. Half the panelists would use the test product first and the other half would use the comparative product first.
3) At the end of the test, the panelists rate their preference (on a 0-5 point scale) on the attribute of “exfoliation”.

The degree of exfoliation is defined as the consumer rating on the 0-5 point scale

d. Skin Abrasiveness Test

Skin abrasiveness is defined as consumer rated response of abrasivity on a 0-9 scale (0 means no abrasion, 10 is abrasivity caused by a pouf (i.e. a showering implement composed of thin plastic filaments, see also e.g. U.S. Pat. No. 5,650,384 to Gordon et al.).

This test is performed with 50 untrained consumers. They are asked to rate the abrasiveness of the test product on a 0-9 point scale. The data is normalized based on their response to a bar with no exfoliants which is assigned a value of zero and a pouf that is assigned a value of 9. The test products are applied to the flex area of the forearm by wetting the bar and rubbing back and forth 10-15 times.

e. Skin Smoothness

Skin smoothness is evaluated (clinically) via Primos® (in-vivo optical skin topography measuring device supplied by GFM Esstezhnik GmbH, Berlin, Germany). Baseline roughness is measured (on leg/arms—starting dryness around grade 1-2). For wash, bar rubbed on skin for 30 secs and the lather left on for 90 secs, rinsed for 30 secs at 35 C. Measure again the roughness 30 minutes after wash process. This procedure may be performed twice a day for a period of up to 5 days.

Smoothness is defined as the mean decrease in roughness at end of study period. Alternately skin smoothness can also be evaluated in a consumer test as follows:

The consumer test protocol consists of:
1) Recruiting approx. 10-20 women in the age group of 25-65 and who are complexion bar users.
2) Use test and comparative products for a week each. Half the panelists would use the test product first and the other half would use the comparative product first.
1) At the end of the test, the panelists rate their preference (on a 0-5 point scale) on the attribute of “Skin feels smoother”. Smoothness is defined as the consumer rating on the 0-5 point scale

f. Skin Softness

Skin softness may be evaluated using the Linear Skin Rheometer (Goodyear Scientific Instruments, UK). Exfoliated skin has less dry flakes—hence is more soft/less stiff. The test involves baseline skin rheometer readings (on the leg/arms) to measure the dynamic spring constant (mgf/mm) of skin which is related to skin stiffness/softness. For wash, the bar is rubbed on the skin for 30 secs and the lather left on for 90 secs, rinsed for 30 secs (at a suitable temperature e.g. 35 C), and the skin is gently pat dry. Next measure skin stiffness/softness 30 minutes after wash. This procedure may be performed twice a day for a period of up to 5 days. Softness is defined as the mean decrease in dynamic spring constant during the study period observed during the study period.

Alternately skin softness can also be evaluated in a consumer test as follows:

The test protocol consists of
1) Recruiting approx. 10-20 women in the age group of 25-65 and who are complexion bar users.
2) Use test and comparative products for a week each. Half the panelists would use the test product first and the other half would use the comparative product first.
3) At the end of the test, the panelists rate their preference (on a 0-5 point scale) on the attribute of “Skin feels softer”.

Softness is defined as the consumer rating on the 0-5 point scale

g) Zein Test Method

The cleansing base of the inventive toilet bar preferably have zein solubilities of under about 50, 40, 30, and most preferably under about 25 using the zein solubility method set forth below. The lower the zein score, the milder the product is considered to be. This method involves measuring the solubility of zein (corn protein) in cleansing base solutions as follows:

0.3 g of cleansing base and 29.7 g of water are mixed thoroughly. To this is added 1.5 g of zein, and mixed for 1 hour. The mixture is then centrifuged for 30 minutes at 3000 rpm. After centrifugation, the pellet is extracted, washed with water, and dried in a vacuum oven for 24 hours until substantially all the water has evaporated. The weight of the dried pellet is measured and percent zein solubilized is calculated using the following equation:
% Zein solubilized=100 (1-weight of dried pellet/1.5).

The % Zein is further described in the following references: E. Gotte, Skin compatibility of tensides measured by their capacity for dissolving zein protein, Proc. IV International Congress of Surface Active Substances, Brussels, 1964, pp 83-90.

h) Bar Sensory Exfoliation Index

The bar sensory exfoliation index is determined using the following procedure: The user takes the bar in one hand and rotates it under running water at 35 C. The number of rotations required for the exfoliant to be perceived (i.e. by tactile sensation) by the user is recorded. The bar exfoliation index is defined as the mean number of rotations required to perceive the exfoliant particles in the bar.

i) General Consumer Test Protocol

The test protocol consists of
1) Recruiting approx. 10-20 women in the age group of 25-65 and who are complexion bar users.
2) Use test and comparative products for a week each. Half the panelists would use the test product first and the other half would use the comparative product first.
2) At the end of the test, the panelists rate their preference on a 0-5 point scale for the following attributes:
   Exfoliates
   Provides Gentle Exfoliation
   Moisturizes and exfoliates
   Skin feels softer
   Skin feels smoother
   Is good for dry skin

While this invention has been described with respect to particular embodiments thereof, it is apparent that numerous other forms and modifications of the invention will be obvious to those skilled in the art. The appended claims and this invention generally should be construed to cover all such obvious forms and modifications which are within the true spirit and scope of the present invention.

1. A massaging toilet bar, comprising:
a. about 5 to 80% by wt. of one or more anionic surfactants;
b. about 0.02 to 20% by wt. of disintegrable agglomerates, the agglomerates including a plurality of particles, optionally containing a binder; and a treatment fluid coating at least a portion of an interior surface of the particles in the agglomerates, the fluid being selected from a hydrophilic compound, a hydrophobic compound or a blend thereof;
c. wherein the agglomerates have a fracture index less than 1.0; and
d. wherein the bar has a rigidity index of greater than 0.2 Mpa at 25 C.

2. The bar of claim 1 wherein the disintegrable agglomerates are disintegrable in water or any other hydrophilic substance with or without applied shear force.

3. The bar of claim 1 wherein the average weight ratio of treatment fluid to particles and optional binder in the agglomerates is in the range of about 1:5 to about 5:1.

4. The bar of claim 1 where the average diameter of the agglomerates is in the range of about 0.1 to 20.0 mm.

5. The bar of claim 1 wherein the treatment fluid is in an effective viscosity range for the fluid to at least partially permeate the agglomerates and coat at least a portion of the interior surface of the particles in the agglomerates prior to the bar becoming solidified.

6. The bar of claim 1 wherein the treatment fluid is selected from polyhydric alcohols, fatty acids; polyols; tri and diglyceride oils; petroleum oils; silicone oils, fluorinated oils, brominated oils, C14 and lower alkyl esters; C14 and lower alkyl alcohols; or blends and derivatives thereof.

7. The bar of claim 1 wherein the treatment fluid has less than about 10% of water.

8. The bar of claim 1 wherein the plurality of particles is agglomerated prior to being formulated into the bar, is agglomerated during bar formulation, or is agglomerated using a combination thereof.

9. The bar of claim 1 wherein the plurality of particles are selected from hydrophilic clays; hydrophobically modified clays; silica; zeolite; cellulose; starch; or blends and derivatives thereof.

10. The bar of claim 1 wherein the one or more anionic surfactants contains
a. about 0 to 30% by wt. of one or more fatty acid soaps; and
b. about 15 to 60% by wt. of one or more non-soap anionic surfactants.

11. The bar of claim 1 wherein the one or more anionic surfactants contains
a. about 30 to 80% by wt. of one or more fatty acid soaps; and
b. about 5 to 40% by wt. of one or more non-soap anionic surfactants.

12. The bar of claim 1 wherein the one or more anionic surfactants contains
a. about 30 to 80% by wt. of one or more fatty acid soaps; and
b. about 0 to 10% by wt. of one or more non-soap anionic surfactants.

13. A process for manufacturing a massaging bar, comprising the steps of in no particular order:
a. contacting particle agglomerates with a treatment fluid under conditions effective for the agglomerate to have attained a fracture index of less than 1.0 to form a pretreated agglomerate, the fluid being selected from a hydrophilic compound, a hydrophobic compound or a blend thereof;
b. blending the pretreated agglomerate with at least one component of a toilet bar base selected from a soap base, a syndet base, or a combar base, optionally containing a fragrance and other minor components to form a mixed mass;
c. treating the mixed mass via an effective combination of milling, refining and/or plodding to form a refined mass; and
d. processing the refined mass by either extruding followed by stamping, cutting or a combination thereof, or casting followed by hardening to form a massaging bar.

14. A method of skin cleansing with a massaging bar, comprising the steps of:
a. wetting the massaging bar with water; the bar including:
1. about 5 to 80% by wt. of one or more anionic surfactants;
2. about 0.02 to 20% by wt. of water-disintegrable agglomerates, the agglomerates including a plurality of particles, optionally containing a binder; and a treatment fluid coating at least a portion of an interior surface of the particles in the agglomerates, the fluid being selected from a hydrophilic compound, a hydrophobic compound or a blend thereof;
3. wherein the agglomerates have a fracture index less than 1.0; and
4. wherein the bar has a rigidity index of greater than 0.2 Mpa at 25 C; and
b. rubbing the wet bar on the skin with sufficient force to cause the agglomerates to disintegrate while cleansing and massaging the skin.
